(19) United States
(12) Patent Application Publication
(10) Pub. No.: US 2026/0345493 A1
(43) Pub. Date: Nov. 5, 2026

(54) POWDERY SUPPORTED MCT-COMPOSITIONS AND THEIR USE

(71) Applicant: IOI OLEG GMBH, Hamburg (DE)

(72) Inventors: Sebastian REYER, Hamburg (DE); Sven STEPAN, Hamburg (DE)

(21) Appl. No.: 19/158,874

(22) PCT Filed: Mar. 15, 2023
(86) PCT No.: PCT/EP2023/056610

§ 371 Date: Aug. 21, 2025

(30) Foreign Application Priority Data
Feb. 24, 2023 (WO) PCT/EP2023/054759
Mar. 9, 2023 (WO) PCT/EP2023/055982

(51) Int. Cl. A61K 9/16; A61K 8/37; A61K 8/73

(52) U.S. Cl. CPC A61K 9/1617; A61K 8/375; A61K 8/73; A61K 9/1652

(57) ABSTRACT

The present invention relates to specifically particulate or powdery supported compositions containing medium-chain triglycerides (MCTs) and their uses, and to processes for their production.

### CROSS-REFERENCES TO RELATED APPLICATIONS

This application is a National Stage filing of international Application PCT/EP 2023/056610 (WO 2024/175213) filed Mar. 15, 2023, entitled “POWDERY SUPPORTED MCT-COMPOSITIONS AND THEIR USE” claiming priority to PCT/EP 2023/054759 filed Feb. 24, 2023, and PCT/EP 2023/055982 filed Mar. 9, 2023. The subject application claims priority to PCT/EP 2023/054759, PCT/EP 2023/055982 and PCT/EP 2023/056610 and incorporates all by reference herein, in their entirety.

### BACKGROUND OF THE INVENTION

The present invention relates to the technical field of medium-chain triglycerides (synonymously also referred to as MCTs) and, in particular, to the provision of such triglycerides in specific compositions.

In particular, the present invention relates to a composition, in particular a particulate composition, which comprises medium-chain triglycerides (MCTs) and which is preferably in powder form, in particular for use in food or foodstuff, cosmetic or pharmaceutical products, wherein the composition comprises or consists of a plurality of individual particles and wherein the particles comprise at least one polysaccharide-based carrier (i.e. support) material based on gum arabic.

Furthermore, the present invention also relates to related food, cosmetic or pharmaceutical products, each comprising the composition according to the invention.

Furthermore, the present invention also relates to the use of the composition according to the invention in a food, cosmetic or pharmaceutical product or for the preparation of a food, cosmetic or pharmaceutical product.

Furthermore, the present invention also relates to a process for the preparation of the composition according to the invention.

The present invention also relates to a process for controlling or adjusting the loading of a polysaccharide-based carrier material comprising or formed from gum arabic with at least one medium-chain triglyceride (MCT), in particular for the preparation of a particulate or powder composition comprising medium-chain triglycerides (MCTs), as defined according to the invention.

Finally, the present invention also relates to the use of the glycoprotein content of a polysaccharide-based carrier (i.e.support) material comprising or formed from gum arabic for controlling or adjusting the loading of the polysaccharide-based carrier material with at least one medium-chain triglyceride (MCT).

In the prior art, oils and fatty phases or fats (e.g. triglycerides or the like) are often used, particularly in the food sector and also in the cosmetic and pharmaceutical sectors, to provide the products or articles with special properties. This is usually done on the basis of the physical or physicochemical properties of the substances used and their nutritional or metabolic relevance, particularly in the case of foodstuffs. However, there is often the problem that oils or fat phases in their pure form cannot always be satisfactorily incorporated into the corresponding products.

In this context, it is often problematic to effectively incorporate oils or fat phases into corresponding products in order to obtain high contents on the one hand and stable or homogeneous emulsions on the other. It is often not possible to emulsify oils or fat phases homogeneously and stably. In particular, a component or system segregation or the like can occur as a result, accompanied by insufficient (storage) stability and ultimately limited product quality. In this respect, it is not possible to specify or permanently maintain the corresponding product properties to a sufficient extent. In addition, the bioavailability of the underlying components is often lower.

Against this background, there is therefore a great overall demand in the state of the art for the provision of relevant products or products, in particular in the form of homogeneous and stable dispersions, in particular emulsions, in which a stable, homogeneous and permanent incorporation of the oils or fat phases is given, whereby high contents or concentrations of oils or fats can also be realized.

Against this background, approaches are being pursued in the state of the art to apply, fix or encapsulate the oils or fat phases to be used in corresponding products or articles on carriers or carrier materials for the purposes of improved handling and improved incorporation. Such supported or encapsulated systems are generally characterized by better handling and processing also with regard to dispersions or emulsions to be produced, compared to the free, in particular non-supported or non-encapsulated oils or fat phases.

As far as the supported or encapsulated oil or fat phase systems are concerned, a large number of usable oils or fat phases and substances for forming carrier systems or encapsulations are generally used.

In addition to numerous other materials, polysaccharide-based carrier materials are used as carrier materials in the prior art, whereby the focus in this context is often on the use of modified starch and maltodextrin or the like. On the basis of such carrier systems, however, a fixation or attachment of corresponding oils or fat phases can often only take place to a certain extent or degree. Such carrier systems are often in the form of particles. The resulting compositions based on the loaded carrier systems do not always have optimum degrees of loading with the oils or fat phases. In addition, the application and processing properties of such compositions based on carrier materials formed from modified starch or maltodextrin are often not always optimal. In addition, the stability is not always good and the flowability or pourability is often less good. In addition, such compositions can also tend to clump or form macroagglomerates, particularly in the case of higher loading levels with the oil or fat phase, as the underlying particulate structures sometimes have a certain stickiness.

In general, the storage stability of prior art carrier systems is often unsatisfactory. In addition, due to the aforementioned disadvantages, supported or encapsulated systems of the prior art often behave in such a way that optimal incorporation or processing into products or articles, for example foodstuffs, cosmetic and pharmaceutical products or articles, is often not possible under the aspect of forming stable and homogeneous dispersions, in particular stable and homogeneous emulsions. This leads to poorer product properties for the resulting products or articles, also with regard to their stability, oil or fat content, bioavailability of the ingredients, application properties or the like.

In addition, particularly in the area of foodstuffs or corresponding products or articles, it should also be noted that carrier materials based on modified starch or maltodextrin are not nutritionally neutral and in this context can also have their own caloric influence, which is problematic or undesirable for dietary food products, for example. Also, the solubility properties are often not optimal when introduced into a liquid, especially water, which is detrimental to the formation of stable or homogeneous emulsions overall.

In addition, gum arabic can also be used as a polysaccharide-based carrier or carrier material. In particular, gum arabic has advantages with regard to emulsifiability. However, with conventional gum arabic used in the prior art, it is generally not possible to achieve the desired high loading quantities with regard to oils or fat phases, as the loading capacity in this respect is generally not sufficient. This means that products based on such compositions or products in which such compositions are incorporated also do not have the desired high quantity or content of oils or fats. To this end, attempts have been made to combine gum arabic with other materials for the carrier system or to use other carrier systems, including, for example, modified starch or maltodextrin as other carrier materials, which in turn is associated with the aforementioned disadvantages.

The scientific publication according to Savihta Krishnan et al. “Microencapsulation of cardamom oleoresin: Evaluation of blends of gum arabic, maltodextrin and a modified starch as wall materials”, Carbohydrate Polymers, Vol. 61, Issue 1, Jul. 4, 2005, pages 95 to 102, concerns the microencapsulation of cardamom oleoresin and the related analysis of blends of gum arabic, maltodextrin and a modified starch. The microencapsulation is formed in particular on the basis of binary and ternary mixtures of the aforementioned components. The focus is therefore on mixing systems for the formation of encapsulations. The use of binary or ternary mixtures is associated with certain disadvantages, as previously mentioned in relation to the respective substances.

As mentioned above, numerous possible carrier materials for oils or fatty phases are used in the prior art.

In this context, U.S. Pat. No. 10,463,626 B2 relates to a dry composition comprising three-dimensional porous microparticles. The microparticles comprise an active ingredient, polymeric nanoparticles, lipid droplets, a nanoparticle stabilizing agent and a cryoprotectant, the active ingredient being carried by the nanoparticles or lipid droplets. The nanoparticles acting as carrier material are based on a PLGA polymer (poly(lactide-co-glycolide)).

Furthermore, the scientific publication according to Vita Paramita et al. “High-Oil-Load Encapsulation of Medium-Chain Triglycerides and d-Limonene Mixture in Modified Starch by Spray Drying”, Journal of Food Science, Vol. 77, Volume 2, pages E38 to E44, concerns the encapsulation of a mixture of medium-chain triglycerides and d-limonene in modified starch. In this respect, modified starch is thus used as a carrier material. As mentioned above, the use of modified starch is associated with certain disadvantages.

In summary, it can therefore be stated for carrier materials of the prior art, which are used for the absorption or fixation of oils or fat phases, as well as for compositions based on such carrier systems, that it is not always possible to realize high degrees of loading with the oils or fat phases and that such systems or compositions are not always optimal for application and use. compositions do not always have optimum application and processing properties, in particular with regard to the provision of corresponding products or articles, such as foodstuffs, pharmaceuticals or cosmetics, and also with regard to the formation of stable or homogeneous emulsions or the like. In addition, the systems or compositions in question do not always have optimum dosing properties.

Due to their special properties, medium-chain triglycerides (MCTs) are of great importance in the food, cosmetic and pharmaceutical product sectors. Medium-chain triglycerides are often used in the production of cosmetics and pharmaceuticals, for example as carriers for other ingredients or components, solubilizers, release agents, lubricants and the like. In addition, medium-chain triglycerides (MCTs) are often used in the food industry, for example due to their metabolic properties or special features, including in dietary foods and the like. For this reason, there is also a high demand for medium-chain triglycerides for the provision of corresponding products or products which are present in the form of homogeneous or stable dispersions, in particular emulsions, whereby a homogeneous distribution or high contents or concentrations as well as a high bioavailability, in particular with regard to food, should be given for the medium-chain triglycerides as such, and this with a high storage stability at the same time. However, with the carrier systems available in the prior art and compositions comprising such carrier systems, a satisfactory finish with medium-chain triglycerides is not always given due to the aforementioned shortcomings.

### BRIEF SUMMARY OF THE INVENTION

Against this background, one object of the present invention is to provide carrier systems or compositions comprising such carrier systems for receiving or fixing oils or fat phases, in particular medium-chain triglycerides (MCTs), which at least largely avoid or at least mitigate the previously described disadvantages of the prior art.

In particular, relevant carrier systems or compositions are to be provided which enable efficient absorption or fixation of oils and fat phases, in particular with regard to medium-chain triglycerides (MCTs), whereby overall high degrees of loading of the carrier systems or the compositions with the medium-chain triglycerides (MCTs) are to be enabled. The relevant carrier systems or compositions should also have improved handling and use or processing properties, in particular with regard to their use and application in further products or articles, which should be in the form of stable and homogeneous dispersions, in particular emulsions, or also in solid or powder form.

In a completely surprising manner, the applicant has discovered in the context of the present invention that a composition provided or loaded with a high content of medium-chain triglycerides (MCTs) can be provided if particles based on a special carrier material, in particular for binding or fixing the medium-chain triglyceride (MCT), are used, namely in the form of a polysaccharide-based carrier material which comprises gum arabic in a special way or is formed therefrom, wherein the gum arabic has a high glycoprotein content, as described in detail below.

Due to the targeted and purposeful use of a carrier material based on a special gum arabic, the finishing or application of the medium-chain triglyceride (MCT) to the carrier material can be improved in a completely surprising way in that large quantities of medium-chain triglyceride (MCT) can be absorbed by the composition or fixed to the particles. According to the invention, a special composition with a high proportion or content of medium-chain triglyceride (MCT) is thus provided, which leads to overall improved product and application properties of the composition, as likewise explained below. Also against this background, the composition according to the invention is suitable for use in a large number of products in the food or foodstuffs sector and in the cosmetics or pharmaceuticals sector.

To solve the object described above, the present invention thus proposes—according to a first aspect of the present invention—a composition, especially a particulate composition, preferably a composition in powder form, preferably a composition in powder form comprising medium-chain triglycerides (MCTs), as described herein; further, in particular special and/or advantageous embodiments of the composition according to the invention are the subject of the the disclosure relating to the composition.

Furthermore, the present invention relates—according to a second aspect of the present invention—to a food, cosmetic or pharmaceutical product comprising a composition according to the invention, further, in particular special and/or advantageous embodiments of this aspect of the invention are provided relating to the product according to the invention.

Similarly, the present invention also relates—according to a third aspect of the present invention—to the use of the composition according to the invention in a food, cosmetic or pharmaceutical product or for the preparation of a food, cosmetic or pharmaceutical product relating to the use of the composition according to the invention; further, in particular special and/or advantageous embodiments of this aspect of the invention are provided.

Furthermore, the present invention relates—according to a fourth aspect of the present invention—to the method according to the invention for producing the composition according to the invention, in particular particulate composition, preferably a composition in powder form, preferably composition in powder form comprising medium-chain triglycerides (MCTs), relating to the method according to the invention; further, in particular special and/or advantageous embodiments of this aspect of the invention are described.

Furthermore, the present invention relates—according to a fifth aspect of the present invention—to the method according to the invention for controlling or adjusting the loading, in particular the amount of loading of a polysaccharide-based carrier material comprising gum arabic or formed therefrom with at least one medium-chain triglyceride (MCT) according to the disclosure; further, in particular and/or advantageous embodiments of this aspect of the invention are provided.

Furthermore, the present invention relates—according to a sixth aspect of the present invention—to the use of the glycoprotein content of a polysaccharide-based carrier material comprising gum arabic or formed therefrom for controlling and/or setting the loading, in particular the amount of loading, of the polysaccharide-based carrier material with at least one medium-chain triglyceride (MCT) according to the disclosure relating to the use according to the invention; further, in particular special and/or advantageous embodiments of the use according to the invention.

In the following explanations, it goes without saying that embodiments, embodiments, advantages and the like, which are explained below for the purpose of avoiding repetition with respect to only one aspect of the invention, naturally also apply correspondingly with respect to the other aspects of the invention, without this requiring separate mention.

Furthermore, it goes without saying that individual aspects and embodiments of the present invention are also deemed to be disclosed in any combination with other aspects and embodiments of the present invention and, in particular, any combination of features and embodiments, as resulting from the back references of all patent claims, is also deemed to be disclosed extensively, namely with regard to all resulting combination possibilities.

In particular, for the features characterizing the invention, any combination of these features is also deemed to be disclosed, whereby embodiments of the same preference of the various features are preferred in their combination (e.g. quantities or quantity ranges of the relevant ingredients of the same preference or the like). Likewise, all other combinations (i.e. combinations based on different preferences or different levels of preference) are also disclosed.

In the case of all relative or percentage weight-related disclosures mentioned below, in particular relative quantity or weight disclosures, it should also be noted that these are to be selected by the skilled person within the scope of the present invention in such a way that these always add up to 100% or 100 wt. % when all components or ingredients are included, in particular as defined below; however, this is self-evident to the skilled person.

In addition, the skilled person may—depending on the application or individual case—deviate from the following values or ranges of the present quantities or contents of the ingredients or components, if necessary, without departing from the scope of the present invention.

In addition, all values or parameters or the like mentioned below can in principle be determined or determined using standardized or standardized or explicitly stated determination methods or otherwise using determination or measurement methods known to a person skilled in the art in this field.

### BRIEF DESCRIPTION OF THE DRAWINGS

FIG. 1 provides a schematic representation or overview based on a flow chart of a process according to the invention for the preparation of a composition (composition P) according to the invention, in particular particulate composition P.

### DETAILED DESCRIPTION OF THE INVENTION

With this proviso made, the present invention will now be explained in detail below:

Subject-matter of the present invention—according to a first aspect of the present invention—is thus the composition according to the invention, preferably composition in powder form, preferably composition in powder form comprising medium-chain triglycerides (MCTs), in particular for use in cosmetic, pharmaceutical or food products,
wherein the composition comprises or consists of a plurality of individual particles,
wherein the particles each comprise at least one polysaccharide-based carrier (i.e. support) material, wherein the polysaccharide-based carrier material is provided and/or loaded with at least one medium-chain triglyceride (MCT), in particular with at least one triglyceride of at least one linear saturated C₆, C₈, C₁₀ or C₁₂ carboxylic acid, and/or wherein the particles each comprise at least one polysaccharide-based carrier material and at least one medium-chain triglyceride bound and/or fixed to the polysaccharide-based carrier material, and/or wherein the particles each comprise at least one polysaccharide-based carrier material and at least one medium-chain triglyceride (MCT) bound and/or fixed to the polysaccharide-based carrier material, in particular at least one triglyceride of at least one linear saturated C₆, C₈, C₁₀ or C₁₂ carboxylic acid bound and/or fixed to the polysaccharide-based carrier material;
wherein the polysaccharide-based carrier material comprises or is formed from gum arabic, wherein the gum arabic comprises a glycoprotein content, based on the gum arabic, of at least 4 wt. % and preferably in the range of 4 wt. % to 25 wt. %, and
wherein the composition or particles comprise a triglyceride content, based on the composition or particles, of at least 50 wt. % and preferably in the range of 50 wt. % to 90 wt. %.

Surprisingly, the applicant has found an efficient and effective way of providing a composition with a high amount or content of medium-chain triglyceride, namely by providing a particulate composition or composition in powder form in a targeted and purposeful manner using a special polysaccharide-based carrier material, which in turn comprises or is formed from a special gum arabic, according to which, in the context of the present invention, the gum arabic has a high glycoprotein content.

In this context, it is equally unexpected and surprising in the context of the present invention that by using gum arabic with a high glycoprotein content, a high amount of medium-chain triglyceride can be bound or fixed to the polysaccharide-based carrier material, while at the same time a high amount of medium-chain triglyceride powdered can be bound or fixed to the polysaccharide-based carrier material. At the same time, the composition according to the invention also has excellent dispersibility, in particular emulsifiability, in liquids, in particular in water, so that products or end products (such as, in particular, food, cosmetic or pharmaceutical products) are obtained on this basis in the form of homogeneous and stable dispersions, in particular emulsions, which also have a high MCT content. The excellent properties with regard to dispersibility, in particular emulsifiability, of the composition according to the invention are also surprising in that a high content of medium-chain triglyceride is fundamentally detrimental to the properties in this respect. The composition according to the invention is also designed to be free-flowing and flowable and can be readily incorporated into compositions in powder form or processed into products, in particular homogeneously and without the use of high mixing and/or shear energy.

In this context, the polysaccharide-based carrier material based on a special gum arabic with a high glycoprotein content used according to the invention is of particular importance. Without wishing to be limited or bound to this theory, the gum arabic used according to the invention acts in particular as an intrinsic dispersant, in particular an emulsifier, with regard to the medium-chain triglycerides, and this in several respects, namely both when loading the composition or the carrier material with the medium-chain triglycerides on the one hand, and when forming the dispersion, in particular emulsion, within the scope of the application or use of the composition according to the invention, with corresponding introduction of the composition into a liquid, in particular water, to form corresponding products on the other hand. In this context—equally without wishing to be bound to limited to this theory—optimal interaction between the components of gum arabic, namely in particular based on medium-chain triglycerides (MCTs) to the heteropolysaccharide-based carrier material and for the subsequent formation of a dispersion, in particular an emulsion, an optimal interaction between the components of gum arabic, namely in particular based on the relevant glycoproteins on the one hand and the medium-chain triglycerides (MCTs) on the other hand. This leads overall to improved binding and loading properties and also to good release properties with regard to the formation of dispersions, in particular emulsions, when the composition is introduced into liquids such as water.

In addition, the particulate composition or composition in powder form according to the invention also exhibits excellent flowability and fluidity. This also goes hand in hand with good manageability in the preparation of the composition according to the invention as well as with good manageability of the composition according to the invention in its application or processing, in particular also with regard to excellent dosability and the avoidance of lump or macroagglomerate formations or the like. On this basis, homogeneous (end) products can be provided on the basis of stable and homogeneous dispersions, in particular emulsions based on the composition according to the invention. In particular, the composition according to the invention surprisingly and despite the high loading or the high content of medium-chain triglycerides (MCTs) does not exhibit any stickiness or adhesiveness of the particles or the like which is contrary to the flowability or pourability. Consequently, the composition according to the invention can in particular also be readily incorporated into compositions in powder form or processed into products in powder form, in particular homogeneously and without the use of large mixing and/or shearing energy.

In particular, on the basis of the composition according to the invention, corresponding (end) products, such as food, cosmetic or pharmaceutical products, can be provided with a high content of medium-chain triglycerides (MCTs), which is accompanied by further central advantages, namely in particular as a result of the positive properties of medium-chain triglycerides (MCTs), for example as carriers, solubilizers, release agents, lubricants, hydrophobizing agents, film and protective formers and viscosity regulators or the like. On this basis, corresponding products with optimized properties can be provided.

In addition, medium-chain triglycerides have excellent nutritional and metabolic properties, so that the relevant properties of corresponding (food) products can also be improved.

Overall, on the basis of the composition according to the invention, the incorporation of medium-chain triglycerides (MCTs) into formulations to obtain corresponding products or products can be facilitated or improved.

As a result of the provision of corresponding products or articles with a high content of medium-chain triglycerides (MCTs), good care properties and application properties (e.g. good application of creams or the like to the skin) can be provided, for example with regard to cosmetics.

In this context, too, the medium-chain triglycerides (MCTs) have a high bioavailability, in particular because they are homogeneously distributed in the ready-to-use products and incorporated in stable dispersions, in particular emulsions.

The composition according to the invention also has the advantage that it can in particular be vegan. In addition, the composition according to the invention can be at least substantially tasteless and/or odorless or at least substantially colorless. In particular, the composition according to the invention also has a high stability, in particular storage stability. In this context, the compositions according to the invention can have a storage stability of at least 36 months at a storage temperature below 25 °C in a dry atmosphere (i.e. relative humidity of less than 60%), in particular when stored or kept in an airtight and/or moisture-proof and/or opaque container.

In addition, it is also the case in the context of the present invention that the composition according to the invention also has an associated high keto body index (ketogenic index) as a result of the high content of medium-chain triglycerides (MCTs), so that corresponding products or products with an equally high keto body index can also be provided on this basis. Against this background, the composition according to the invention also has a correspondingly high nutritional-physiological or dietary relevance, for example with regard to the use of the composition according to the invention in foodstuffs or dietary foodstuffs or the like. This is because during physiological metabolism of medium-chain triglycerides (MCTs) in the human body, keto or ketone bodies are formed, which is of corresponding nutritional-physiological relevance, for example with regard to diets, special nutrition or the like. In particular, a high energy density of relevant compositions and products or articles can also be provided on this basis.

The composition according to the invention can be used, for example, in ointments, creams, pastes, lotions, gels, sprays or liquids, as will be mentioned below. The composition according to the invention can in particular also be used for products or products based on dry formulations, in particular formulations in powder form, especially since good miscibility is also present. In particular, use for dry or powdered cosmetic products, such as shampoos, shower gels, skin care products or the like, may be appropriate. However, formulations in powder form or products based on dry formulations, in particular formulations in powder form, can also be considered for the area of food and food products as well as pharmaceuticals.

If necessary or desired, liquid, in particular water, can be added by the user. Due to the excellent dispersibility, in particular emulsifiability, the addition of a liquid, in particular water, results in the immediate formation of a homogeneous dispersion, in particular emulsion, ready for use.

In this context, the composition according to the invention also has the advantage that an emulsion is obtained immediately after the addition of a liquid, in particular water (or solvents), even without further heating. In this context, the composition according to the invention is thus in particular also an effective transformation composition or powder which, after addition of liquid, in particular water, leads to a stable and homogeneous emulsion (“from powder to emulsion”). On this basis, the present invention facilitates the incorporation or incorporation of MCTs into products, also for surfactant-comprising formulations (“from powder to surfactant”). The composition according to the invention can also be added in particular to formulations in powder form, such as particle- or powder-form and/or essentially water-free skin and hair cleansing products, in particular (dry) shampoos or (dry) shower gels. In particular, the composition according to the invention can also be added to formulations based on balms, spray-dried emulsions or mixtures of different oils. A high content of MCTs can also be set in such formulations, which also leads to improved care properties in cosmetic products, also with regard to powder formulations. In this context, the disadvantage can be overcome in the context of the present invention that it is often not possible to set a high oil or fat phase content, particularly in the case of particle or powder formulations or products that are at least essentially anhydrous. Against this background, the present invention also represents a valuable contribution to the development or provision of products or articles, such as skin care products or the like, in which at least essentially no water is used in the manufacture and in which water is only added during application or use by the consumer to form an emulsion, which can also take place at room temperature due to the excellent properties. This is also conducive to sustainability. Corresponding emulsions can be formed on this basis, which can be applied to the skin, for example, and have good care properties in this respect. In particular, high contents of MCTs can be realized in this context, as mentioned above.

In summary, it should therefore be emphasized that the concept according to the invention with the use of a special gum arabic to provide a corresponding polysaccharide-comprising carrier material for medium-chain triglycerides (MCTs) leads in a surprising way to a high loading with medium-chain triglycerides (MCTs), namely in the case of the compositions according to the invention to a triglyceride content of at least 50 wt. %. In the compositions according to the invention and this with simultaneously excellent flowability and formation of stable dispersions or emulsions in the context of the use of the composition according to the invention. The gum arabic used according to the invention has a high content of glycoproteins of at least 4 wt. % and preferably in the range from 4 wt. % to 25 wt. %, based on the gum arabic.

As far as the particles based on the polysaccharide-based carrier material and the at least one medium-chain triglyceride (MCT) are concerned, these can be present in any form or any physical configuration. In particular, the particles can be formed as matrix particles, in particular where the carrier material forms the matrix for holding the medium-chain triglycerides (MCTs). In this respect, the medium-chain triglycerides (MCTs) can be embedded or incorporated in the matrix or carrier material and/or deposited on the matrix or carrier material; in principle, the matrix can be porous or continuous or dense. In addition, the particles based on the polysaccharide-based carrier material and the at least one medium-chain triglyceride (MCT) can also be formed as capsule particles, in particular core/shell capsules (core/shell structure or core/shell structure). In this respect, it may in particular be envisaged that the polysaccharide-based carrier material forms the capsule shell which encloses the medium-chain triglycerides (MCTs) (whereby additional medium-chain triglycerides may also be present on or at the outer capsule surface).

According to a preferred embodiment of the present invention, it may be provided that the particles of the composition according to the invention each comprise and/or are formed from a plurality of primary particles (=base particles), in particular wherein the primary particles are bonded and/or agglomerated with each other and/or in particular wherein the primary particles each comprise or are formed from the at least one medium-chain triglyceride and the gum arabic, preferably each with core/shell-structure and/or preferably each as core/shell primary particles, preferably wherein the core comprises or is formed from the at least one medium-chain triglyceride and the shell comprises or is formed from the gum arabic. According to a preferred embodiment, in particular in the case of high MCT loadings, it may in particular optionally be provided that the at least one medium-chain triglyceride is additionally present on or at the surface of the core/shell structure and/or the core/shell primary particles, in particular on the shell (i.e. as a so-called “free” surface oil, the proportion or content of which can be determined analytically, in particular by solvent extraction with a generally lipophilic solvent such as diethyl ether); in this context, the content of medium-chain triglyceride(s) present on or at the surface of the core/shell structure and/or the core/shell primary particles, in particular on the shell, based on the particles, in particular 5 wt. % to 60 wt. %, preferably 10 wt. % to 55 wt. %, preferably 15 wt. % to 50 wt. %, particularly preferably 20 wt. % to 45 wt. %.

According to a preferred embodiment of the present invention, it may further be provided that the particles of the composition according to the invention each comprise and/or are formed from a plurality of primary particles (=base particles) connected and/or agglomerated with each other, in particular wherein the primary particles each comprise or are formed from the at least one medium-chain triglyceride and the gum arabic, preferably each with a core/shell structure and/or preferably each as core/shell primary particles, preferably wherein the core comprises or is formed from the at least one medium-chain triglyceride and the shell comprises or is formed from the gum arabic. According to a preferred embodiment, in particular in the case of high MCT loadings, it may in particular optionally be provided that the at least one medium-chain triglyceride is additionally present on or at the surface of the core/shell structure and/or the core/shell primary particles, in particular on the shell (i.e. as a so-called “free” surface oil, the proportion or content of which can be determined analytically, in particular by solvent extraction with a generally lipophilic solvent such as diethyl ether); in this context, the content of medium-chain triglyceride(s) present on or at the surface of the core/shell structure and/or the core/shell primary particles, in particular on the shell, based on the particles, in particular 5 wt. % to 50 wt. %, preferably 10 wt. % to 40 wt. %, preferably 15 wt. % to 30 wt. %.

With regard to the gum arabic used according to the invention, this also has further improved properties as an emulsifier and stabilizer, also in relation to the medium-chain triglycerides (MCTs) as used according to the invention. Furthermore, gum arabic is generally well tolerated, as it is non-toxic and non-allergenic. In addition, gum arabic is physiologically inert, whereby at least essentially no metabolism occurs upon absorption into the body, so that, for example, no additional calories are absorbed in the context of use for food products or the like and, moreover, the keto metabolism associated with the MCTs is not disturbed. This is particularly beneficial with regard to keto body formation. In addition, gum arabic, as used according to the invention, is a vegan substance or a natural-based raw material with a defined composition.

In general, gum arabic (synonymously also called Arabic gum) refers to the gum from the exudate of various acacia trees, which are particularly widespread in Africa.

In general, gum arabic comprises polysaccharides, particularly on the basis of acidic alkali or alkaline earth salts of arabic acid or polyarabic acid. In addition, gum arabic also comprises glycoproteins (in particular arabinogalactan proteins and also other glycoproteins, i.e. glycoproteins other than arabinogalactan proteins).

In general, gum arabic is obtained from the exudate or wound sap of the trees, as mentioned above. In particular, this can be done by cutting into the peeled tree bark at a downward angle. Over the course of 20 to 30 days, a hard blister or worm-shaped or horn-shaped pieces can form from the oozing gum, which can then be removed or harvested. However, the tree can also shed gum on its own, especially if the bark cracks due to drying out. The resulting material can be dried further and subjected to further processing steps, such as purification steps or the like.

As will be explained in more detail below, the present invention according to one embodiment of the invention relates in particular to the use of a very special gum arabic, which is obtained in particular from the gum arabic tree (Senegalia senegal or synonymously Acacia senegal or Senegal acacia) or originates therefrom. In this connection, the applicant has surprisingly found that such a special gum arabic has a correspondingly high content of glycoproteins and is therefore particularly suitable as a polysaccharide-based carrier material for the composition according to the invention.

Gum arabic is described or characterized in particular by the CAS number (CAS registration number) 9000-01-5.

In the European Union, gum arabic is approved as a food additive with the E number E 414.

For further details on gum arabic, please also refer to Römpp Chemie Lexikon, 10th edition, Volume 2, Georg Thieme Verlag, Stuttgart/New York, 1997, pages 1625-1626, keyword: “Gummi arabicum”, as well as the literature referenced there.

The glycoproteins mentioned, as they are provided by gum arabic or as they are present in gum arabic (in particular naturally), are generally (macro)molecules which are based on a protein or protein residue or group with one or more carbohydrate group(s) (i.e. sugar groups) bound to it, in particular covalently bound. In other words, the glycoproteins as provided by gum arabic are generally glycosylated proteins, i.e. proteins or protein residues or groups bound to carbohydrate groups or residues (i.e. sugar groups or residues). In principle, the carbohydrate groups or residues can vary greatly and range from mono- to di- and oligosaccharides to polysaccharides, whereby, in the context of the present invention, oligosaccharide and, in particular, polysaccharide groups are present as carbohydrate groups of the respective glycoproteins.

In particular, the term glycoproteins as used in the context of the present invention refers to the totality of all glycoproteins present. In other words, the term glycoproteins as used in the context of the present invention is used as a generic or collective term for all glycoproteins present in the gum arabic used according to the invention. This thus includes in particular arabinogalactan proteins as specific glycoproteins present in the gum arabic used according to the invention, but also all other glycoproteins present in the gum arabic used according to the invention (i.e. also glycoproteins other than arabinogalactan proteins).

Consequently, the term glycoprotein content of the gum arabic used according to the invention (i.e. the content of glycoproteins in the gum arabic used according to the invention), as used in the context of the present invention, refers to the total content of all glycoproteins present in the gum arabic used according to the invention.

The glycoprotein content of the gum arabic used according to the invention also refers to the respective glycoprotein molecule as a whole or as such (i.e. not only to its pure content of protein(s) or protein groups).

The glycoprotein content of the gum arabic used according to the invention can be determined by conventional methods of analysis or determination known or familiar to the person skilled in the art, in particular chromatographically, preferably by HPLC chromatography or SEC method, in particular SEC-MALLS method (Size-Exclusion Chromatography coupled to Multi-Angle Laser-light Scattering). This is well known to the skilled person. In this regard, reference can be made to the relevant technical literature and specialist publications, in particular L. Lopez-Torrez et al. “Acacia senegal vs. Acacia seyal gums—Part 1: Composition and structure of hyperbranched plant exudates”, Food Hydrocolloids, Volume 51, October 2015, pages 41 to 53 (cf. also URL or link: https://doi.org/10.1016/j.foodhyd.2015.04.019), as well as the literature referenced therein (see in particular L. Picton et al. “Analysis of a complex polysaccharide (gum arabic) by multi-angle laser light scattering coupled on-line to size exclusion chromatography and flow field flow fractionation”, Carbohydrate Polymers, Volume 42, Issue 1, May 2000, pages 23 to 31; see also URL or link: https://doi.org/10.1016/S0144-8617(99)00139-3), the aforementioned references and the entire disclosure thereof relating to the determination of glycoprotein content being hereby incorporated by reference in their entirety.

For the sake of understanding and clarity only, the following is also pointed out in this context: The characterizing parameter of the glycoprotein content used according to the invention is to be distinguished from the parameter of the protein content (whereby the protein content, in particular in the food sector, is determined via the determination of the total nitrogen content with subsequent conversion via a nitrogen conversion factor [typically 6.25 or 6.38], whereby this protein content is generally significantly lower than the glycoprotein content parameter used according to the invention, since only one and sometimes only a relatively small proportion of the glycoprotein molecules are formed from protein groups, whereas the remaining and often predominant proportion of the molecules is formed by carbohydrate or saccharide residues or groups). The determination of the protein content is also based on an independent determination method: For further details on the determination of the protein content, reference is made to: DIN EN ISO 8968-2, June 2002 edition, and to the official collection of methods for sampling and testing foodstuffs, consumer goods, cosmetics, tobacco products and animal feed (ASU): BVL L 01.00-10/2:2016-03, Analysis of foodstuffs—Determination of nitrogen content in milk—Part 2: Block digestion method (macro method) (adoption of the German standard of the same name DIN EN ISO 8968-2, June 2002 edition); see also “IDF Fact Sheet on Protein Determination” (Verband der Deutschen Milchwirtschaft e.V.—VDM), May 2013 edition.

In general, the glycoproteins present in the gum arabic used according to the invention have a weight-average molecular weight M̅_{w} in the range from 10⁴ g/mol to 10⁷ g/mol, in particular in the range from 1×10⁵ g/mol to 9×10⁶ g/mol, preferably 1.5×10⁵ g/mol to 6×10⁶ g/mol. This makes it possible to achieve particularly good properties, in particular application properties, of the composition according to the invention and high MCT loadings.

In general, it may be provided according to the invention that the glycoprotein content of the gum arabic is at least partly, in particular predominantly, provided and/or formed by arabinogalactan proteins (synonymously also referred to as “AGPs”). In this way, particularly good properties, in particular application properties, of the composition according to the invention and high MCT loadings can be achieved.

According to the invention, it is particularly preferred if at least 50%, in particular at least 60%, preferably at least 65%, of the glycoprotein content of the gum arabic is provided and/or formed by arabinogalactan proteins, based on the glycoprotein content.

Furthermore, according to the invention, it is particularly preferred if the glycoprotein content of the gum arabic is provided and/or formed by 50% to 99%, in particular 60% to 98%, preferably 65% to 95%, by arabinogalactan proteins (synonymously also referred to as “AGPs”), based on the glycoprotein content.

The medium-chain triglycerides (MCTs or medium-chain triglycerides or MCTs) are generally triglycerides that comprise medium-chain fatty acids. Medium-chain fatty acids include caproic, caprylic, capric and lauric acid saturated fatty acids. The aforementioned acids are saturated straight-chain or unbranched C₆, C₈, C₁₀ or C₁₂ carboxylic acids. Medium-chain triglycerides can be obtained on an industrial scale, for example by hydrolysis of vegetable fats, such as coconut fat and palm kernel oil, fractionation of the medium-chain fatty acids and subsequent esterification with glycerol. The medium-chain triglycerides (MCTs) used according to the invention are generally, in particular, at least essentially colorless and also odorless and tasteless.

For further information on medium-chain triglycerides (MKTs or MCTs), reference can also be made to Römpp Chemie Lexikon, 10th edition, volume 4, Georg Thieme Verlag, Stuttgart/New York, 1998, page 1560, keyword “MCT (MKT)”, as well as the literature referred to therein in each case.

In the following, the composition according to the invention is described in further detail with reference to further embodiments and embodiments of the present invention:

As previously stated, a special gum arabic with a high glycoprotein content is used in the context of the present invention. As stated above, this allows a particularly high loading of the underlying polysaccharide-based carrier material with medium-chain triglycerides (MCTs) to be realized.

In the context of the present invention, it can be in particular as follows with regard to the glycoprotein content:
The gum arabic may have a glycoprotein content, based on the gum arabic, of at least 4.5 wt. %, in particular at least 5 wt. %, preferably at least 5.5 wt. %, more preferably at least 6 wt. %.
In addition, the gum arabic may have a glycoprotein content, based on the gum arabic, of at most 25 wt. %.
Furthermore, the gum arabic may have a glycoprotein content, based on the gum arabic, of at most 22 wt. %, in particular at most 20 wt. %, preferably at most 18 wt. %, preferably at most 16 wt. %.
According to the invention, it may in particular be provided that the gum arabic has a glycoprotein content, based on the gum arabic, in the range from 4.5 wt. % to 22 wt. %, in particular in the range from 5 wt. % to 20 wt. %, preferably in the range from 5.5 wt. % to 18 wt. %, preferably in the range from 6 wt. % to 16 wt. %.

As stated above, particularly high loadings of the composition or the particles with the underlying polysaccharide-based carrier material can be obtained according to the invention, in particular as a result of the high glycoprotein content.

The triglyceride content according to the invention, as it is stated for the composition according to the invention, refers in the present case to the content of the at least one medium-chain triglyceride, in particular the content of the at least one triglyceride of at least one linear saturated C₆, C₈, C₁₀ or C₁₂ carboxylic acid.

In this respect, it can be in particular as follows according to the invention:
Thus, the composition or the particles may have a triglyceride content, based on the composition or the particles, of at least 55 wt. %, in particular at least 57.5 wt. %, preferably at least 60 wt %, preferably at least 65 wt. %.
In particular, the composition or the particles may have a triglyceride content, based on the composition or the particles, of at most 90 wt. %.
Further, the composition according to the invention or the particles, based on the composition or the particles, may have a triglyceride content of at most 87.5 wt. %, in particular at most 85 wt. %, preferably at most 82.5 wt. %, preferably at most 80 wt. %.
In particular, it may be provided according to the invention that the composition or the particles, based on the composition or the particles, has or have a triglyceride content in the range from 55 wt. % to 87.5 wt. %, in particular in the range from 57.5 wt. % to 85 wt. %, preferably in the range from 60 wt. % to 82.5 wt. %, preferably in the range from 65 wt. % to 80 wt. %.

In addition, the composition according to the invention or the particles of the composition may have the following contents of gum arabic:
It may be provided according to the invention that the composition or the particles, based on the composition or the particles, has or have a gum arabic content of at most 50 wt. %, preferably in the range from 10 wt. % to 50 wt. %.
In addition, it may also be provided in the context of the present invention that the composition or the particles, based on the composition or the particles, has or have a gum arabic content of at most 45 wt. %, in particular at most 42.5 wt. %, preferably at most 40 wt. %, preferably at most 35 wt. %.
In particular, it may be provided according to the invention that the composition or the particles, based on the composition or the particles, has or have a gum arabic content in the range from 12.5 wt. % to 45 wt. %, in particular in the range from 15 wt. % to 42.5 wt. %, preferably in the range from 17.5 wt. % to 40 wt. %, preferably in the range from 20 wt. % to 35 wt. %.

Furthermore, the following combinations of contents or amounts of the respective ingredients or components of the composition according to the invention are also of great importance. In particular, the composition according to the invention can also have the following content or amount of medium triglycerides (MCTs) with increasing glycoprotein content of the gum arabic:

Thus, it may be provided according to the invention,
that the gum arabic has a glycoprotein content, based on the gum arabic, of at least 4.5 wt. % and wherein the composition or the particles, based on the composition or the particles, has or have a triglyceride content of at least 55 wt. %; or else
that the gum arabic has a glycoprotein content, based on the gum arabic, of at least 5 wt. % and that the composition or the particles, based on the composition or the particles, has or have a triglyceride content of at least 57.5 wt. %; or else
that the gum arabic has a glycoprotein content, based on the gum arabic, of at least 5.5 wt. % and that the composition or the particles, based on the composition or the particles, has or have a triglyceride content of at least 60 wt. %; or else
that the gum arabic has a glycoprotein content, based on the gum arabic, of at least 6 wt. % and that the composition or the particles, based on the composition or the particles, has or have a triglyceride content of at least 65 wt. %.

In addition, it may be provided according to the invention,
that the gum arabic has a glycoprotein content, based on the gum arabic, of at most 25 wt. % and wherein the composition or the particles, based on the composition or the particles, has or have a triglyceride content of at most 90 wt. %; or else
that the gum arabic has a glycoprotein content, based on the gum arabic, of at most 22 wt. % and wherein the composition or the particles, based on the composition or the particles, has or have a triglyceride content of at most 87.5 wt. %; or else
that the gum arabic has a glycoprotein content, based on the gum arabic, of at most 20 wt. % and wherein the composition or the particles, based on the composition or the particles, has or have a triglyceride content of at most 85 wt. %; or else
that the gum arabic has a glycoprotein content, based on the gum arabic, of at most 18 wt. % and wherein the composition or the particles, based on the composition or the particles, has or have a triglyceride content of at most 82.5 wt. %; or else
that the gum arabic has a glycoprotein content, based on the gum arabic, of at most 16 wt. % and wherein the composition or the particles, based on the composition or the particles, has or have a triglyceride content of at most 80 wt. %.

Further, it may also be provided within the scope of the present invention,
in that the gum arabic has a glycoprotein content, based on the gum arabic, in the range from 4 wt. % to 25 wt. % and wherein the composition or the particles, based on the composition or the particles, has or have a triglyceride content in the range from 50 wt. % to 90 wt. %; or else
that the gum arabic has a glycoprotein content, based on the gum arabic, in the range from 4.5 wt. % to 22 wt. % and wherein the composition or the particles, based on the composition or the particles, has or have a triglyceride content in the range from 55 wt. % to 87.5 wt. %; or else
that the gum arabic has a glycoprotein content, based on the gum arabic, in the range from 5 wt. % to 20 wt. % and wherein the composition or the particles, based on the composition or the particles, has or have a triglyceride content in the range from 57.5 wt. % to 85 wt. %; or else
that the gum arabic has a glycoprotein content, based on the gum arabic, in the range from 5.5 wt. % to 18 wt. % and wherein the composition or the particles, based on the composition or the particles, has or have a triglyceride content in the range from 60 wt. % to 82.5 wt. %; or else
that the gum arabic has a glycoprotein content, based on the gum arabic, in the range from 6 wt. % to 16 wt. % and wherein the composition or the particles has or have a triglyceride content, based on the composition or the particles, in the range from 65 wt. % to 80 wt. %.

As regards the gum arabic used in a preferred manner according to the invention, the following should also be mentioned:

In particular, the gum arabic may be nature-based and/or of natural origin, in particular of plant-based origin.

Furthermore, the gum arabic can be based on or obtained from a plant exudate, in particular based on or obtained from a plant exudate of acacia trees, in particular gum arabic tree (Senegalia senegal or synonymously Acacia senegal or Senegal acacia).

According to the invention, it may in particular be provided that the gum arabic is obtained from acacia trees, in particular the gum arabic tree (Senegalia senegal or synonymously Acacia senegal or Senegal acacia), or originates therefrom. According to the invention, it can also be provided that the gum arabic is obtained from an exudate of acacia trees, in particular the gum arabic tree (Senegalia senegal or synonymously Acacia senegal or Senegal acacia), or is based thereon.

In this context, the applicant has also found in a surprising way that a special gum arabic, which is based on the previously mentioned gum arabic tree (Senegalia senegal or synonymously Acacia senegal or Senegal acacia), is particularly suitable for or as a polysaccharide-based carrier material, especially in view of the fact that the special gum arabic in question has a high glycoprotein content.

According to an embodiment according to the invention, it may in particular be provided that the glycoprotein content of the gum arabic is controlled and/or set by selecting the gum arabic, in particular by selecting the (plant) material or (plant) species used for obtaining the gum arabic, preferably on the basis of an exudate, optionally with additional enrichment, in particular by of fractionation. In this context, the (plant) material or the (plant) species can be selected in particular from the group of acacia trees, in particular gum arabic tree (Senegalia senegal or synonymously Acacia senegal or Senegal acacia).

According to a further embodiment of the present invention, it may likewise be provided that the glycoprotein content of the gum arabic generally used according to the invention is controlled and/or set by enrichment, in particular using fractionation.

The adjustment of the glycoprotein content can also be carried out cumulatively or in addition to the specific selection of gum arabic as previously mentioned.

The gum arabic used in the context of the present invention is also characterized in that the gum arabic comprises, in particular as its main constituent, at least one polysaccharide, preferably at least one polysaccharide comprising preferably acidic alkali metal and/or alkaline earth metal salt(s) of (poly)arabic acid, particularly preferably at least one preferably branched polysaccharide of L-arabinose, D-galactose, L-rhamnose and D-glucuronic acid, in particular in a (molar) ratio in the range from 2.5-4.5:0.5-5:0.1-2:0.5-2.5, preferably in the range of 2.7-4:1-4:0.5-1.5:0.7-2, preferably in the range of 2.9-3.7:1.5-3.5:0.8-1.2:1-1.7, particularly preferably about 3.5:about 2.9:about 1.1: about 1.6, more preferably 3.5:2.9:1.1:1.6 or more preferably 3:3:1:1.

Consequently, according to the invention, in particular a gum arabic is used which has a defined polysaccharide content, also with regard to its specific formation or composition. This further improves the properties compared to the medium-chain triglycerides (MCTs), also with regard to the fixation or application of the MCTs. In particular, the polysaccharides are also associated with a defined behavior of the composition according to the invention or of the particles based on the polysaccharide-based carrier material when dispersed or when an emulsion is formed by introducing the composition into a liquid, in particular water.

In addition, the content of the polysaccharides is also relevant: For example, it may be provided according to the invention that the gum arabic has a polysaccharide content, based on the gum arabic, in the range from 75 wt. % to 96 wt. %. In particular, the gum arabic may have a polysaccharide content, based on the gum arabic, of at least 75 wt. %. In addition, the gum arabic may have a polysaccharide content, based on the gum arabic, of at most 96 wt. %.

In particular, the following may also be provided according to the invention:
Thus, the gum arabic may have a polysaccharide content, based on the gum arabic, in the range from 78 wt. % to 95.5 wt. %, in particular in the range from 80 wt. % to 95 wt. %, preferably in the range from 82 wt. % to 94.5 wt. %, preferably in the range from 84 wt. % to 94 wt. %.
In addition, the gum arabic may have a polysaccharide content, based on the gum arabic, of at least 78 wt. %, in particular at least 80 wt. %, preferably at least 82 wt. %, preferably at least 84 wt. %.
Furthermore, the gum arabic may have a polysaccharide content, based on the gum arabic, of at most 95.5 wt. %, in particular at most 95 wt. %, preferably at most 94.5 wt. %, preferably at most 94 wt. %.

As regards the medium-chain triglycerides (MCTs) used in the context of the present invention, the following may in particular be provided according to the invention:

In particular, the medium chain triglyceride (MCT) may be a triglyceride of a C₈ carboxylic acid, in particular a linear saturated C₈ carboxylic acid. In this context, it may in particular be provided that the medium-chain triglyceride (MCT) is tricaprylin.

Further, the medium chain triglyceride (MCT) may be a mixed triglyceride of C₈ and C₁₀ carboxylic acids, in particular a mixed triglyceride of linear saturated C₈ and C₁₀ carboxylic acids, respectively. In this respect, the medium-chain triglyceride (MCT) may have a C₈: C₁₀ ratio in the range of 30%-90%: 70%-10%, in particular in the range of 50%-80%: 50%-20%, preferably in the range of 55%-75%: 45%-25%.

The above-mentioned medium-chain triglycerides (MCTs) lead to particularly good application and handling properties, also with regard to products or articles in the cosmetic, pharmaceutical or food sector.

In the context of the present invention, compositions with a high keto body index can be provided as a result of the high loading with MCTs. The keto body index can also be used as a measure of the loading or equipment of the composition according to the invention with the medium-chain triglycerides (MCTs).

The values given below relate in particular to the condition or circumstance that a total of 6 molecules of keto bodies (calculated as 3-hydroxybutyric acid (3-BHB) in the present case) are formed per molecule of a medium-chain triglyceride during physiological metabolism in the human body.

In this context, the physiology is such that medium-chain triglycerides are oxidized, particularly in the liver, releasing keto bodies (ketone bodies), without wishing to refer to or limit this. The keto body index is also based on the excellent nutritional properties of the composition according to the invention. In addition, a high keto body index is also associated with excellent handling and application properties for cosmetic or pharmaceutical products using the composition according to the invention.

Against this background, the following also applies in particular to the keto body index:
According to the invention, the composition according to the invention may have a keto body index (ketogenic index), calculated as the theoretical number of millimoles (mmol) of 3-hydroxybutyric acid (3-BHB) per one gram of the composition, of at least 0.5 mmol/g.
In particular, the composition may have a keto body index (ketogenic index), calculated as the theoretical number of millimoles (mmol) of 3-hydroxybutyric acid (3-BHB) per one gram of the composition, of at least 1 mmol/g, in particular at least 2 mmol/g, preferably at least 3 mmol/g, more preferably at least 4 mmol/g.
According to the invention, the composition according to the invention may have a keto body index (ketogenic index), calculated as the theoretical number of millimoles (mmol) of 3-hydroxybutyric acid (3-BHB) per one gram of the composition, in the range from 0.5 mmol/g to 50 mmol/g.
In particular, the composition may have a keto body index (ketogenic index), calculated as the theoretical number of millimoles (mmol) of 3-hydroxybutyric acid (3-BHB) per one gram of the composition, in the range from 1 mmol/g to 45 mmol/g, in particular in the range from 2 mmol/g to 40 mmol/g, preferably in the range from 3 mmol/g to 30 mmol/g, more preferably in the range from 4 mmol/g to 25 mmol/g.

According to the invention, high values can thus be provided overall for the keto body index of the composition according to the invention. In particular, the composition according to the invention therefore also has a high density or high loading in relation to the medium-chain triglycerides (MCTs). On this basis, corresponding products or products which are provided with the composition according to the invention can also have a high keto body index. This is advantageous both in the cosmetic or pharmaceutical field, in particular with regard to the handling and application properties of the composition according to the invention, and in the food field, in particular with regard to the nutritional properties of the composition according to the invention.

With regard to the composition according to the invention, in particular the following combinations of values for the keto body index, the glycoprotein content of the gum arabic and the triglyceride content can be set or predetermined:

Thus, it may be provided according to the invention,
that the composition comprises a keto body index (ketogenic index), calculated as the theoretical number of millimoles (mmol) of 3-hydroxybutyric acid (3-BHB) per one gram of the composition, of at least 0.5 mmol/g and wherein the gum arabic has a glycoprotein content, based on the gum arabic, of at least 4 wt. % and wherein the composition or particles, based on the composition or particles, comprise a triglyceride content of at least 50 wt. %; or else
that the composition comprises a keto body index (ketogenic index), calculated as the theoretical number of millimoles (mmol) of 3-hydroxybutyric acid (3-BHB) per one gram of the composition, of at least 1 mmol/g and wherein the gum arabic comprises a glycoprotein content, based on the gum arabic, of at least 4.5 wt. % and wherein the composition or the particles, based on the composition or the particles, comprise a triglyceride content of at least 55 wt. %; or else
that the composition comprises a keto body index (ketogenic index), calculated as the theoretical number of millimoles (mmol) of 3-hydroxybutyric acid (3-BHB) per one gram of the composition, of at least 2 mmol/g and wherein the gum arabic has a glycoprotein content, based on the gum arabic, of at least 5 wt. % and wherein the composition or particles, based on the composition or particles, comprise a triglyceride content of at least 57.5 wt. %; or else
that the composition comprises a keto body index (ketogenic index), calculated as the theoretical number of millimoles (mmol) of 3-hydroxybutyric acid (3-BHB) per one gram of the composition, of at least 3 mmol/g and wherein the gum arabic has a glycoprotein content, based on the gum arabic, of at least 5.5 wt. % and wherein the composition or particles, based on the composition or particles, comprise a triglyceride content of at least 60 wt. %; or else
that the composition comprises a keto body index (ketogenic index), calculated as the theoretical number of millimoles (mmol) of 3-hydroxybutyric acid (3-BHB) per one gram of the composition, of at least 4 mmol/g and wherein the gum arabic has a glycoprotein content, based on the gum arabic, of at least 6 wt. % and wherein the composition or the particles, based on the composition or the particles, has or have a triglyceride content of at least 65 wt. %.

In addition, it may also be provided according to the invention,
that the composition has a keto body index (ketogenic index), calculated as the theoretical number of millimoles (mmol) of 3-hydroxybutyric acid (3-BHB) per one gram of the composition, in the range from 0.5 mmol/g to 50 mmol/g and wherein the gum arabic has a glycoprotein content, based on the gum arabic, in the range from 4 wt. % to 25 wt. % and wherein the composition or particles comprise a triglyceride content, based on the composition or particles, in the range of 50 wt. % to 90 wt. %; or else
that the composition has a keto body index (ketogenic index), calculated as the theoretical number of millimoles (mmol) of 3-hydroxybutyric acid (3-BHB) per one gram of the composition, in the range from 1 mmol/g to 45 mmol/g and wherein the gum arabic has a glycoprotein content, based on the gum arabic, in the range from 4.5 wt. % to 22 wt. % and wherein the composition or particles comprise a triglyceride content, based on the composition or particles, in the range of 55 wt. % to 87.5 wt. %; or else
that the composition has a keto body index (ketogenic index), calculated as the theoretical number of millimoles (mmol) of 3-hydroxybutyric acid (3-BHB) per one gram of the composition, in the range from 2 mmol/g to 40 mmol/g and wherein the gum arabic has a glycoprotein content, based on the gum arabic, in the range from 5 wt. % to 20 wt. % and wherein the composition or particles comprise a triglyceride content, based on the composition or particles, in the range of 57.5 wt. % to 85 wt. %; or else
that the composition has a keto body index (ketogenic index), calculated as the theoretical number of millimoles (mmol) of 3-hydroxybutyric acid (3-BHB) per one gram of the composition, in the range from 3 mmol/g to 30 mmol/g and wherein the gum arabic comprises a glycoprotein content, based on the gum arabic, in the range of from 5.5 wt. % to 18 wt. % and wherein the composition or particles comprise a triglyceride content, based on the composition or particles, in the range from 60 wt. % to 82.5 wt. %; or else
that the composition has a keto body index (ketogenic index), calculated as the theoretical number of millimoles (mmol) of 3-hydroxybutyric acid (3-BHB) per one gram of the composition, in the range from 4 mmol/g to 25 mmol/g and wherein the gum arabic has a glycoprotein content, based on the gum arabic, in the range from 6 wt. % to 16 wt. % and wherein the composition or particles comprise a triglyceride content, based on the composition or particles, in the range of 65 wt. % to 80 wt. %.

In addition, it may be provided according to the invention that the composition according to the invention comprises at least one emulsifier.

The use of an emulsifier is associated in particular with the advantage that, on the one hand, the fixation or impingement of the particles of the composition with the medium-chain triglyceride can be further improved. On the other hand, the dispersing or emulsifying behavior of the composition according to the invention is also further improved, in particular when introduced into a liquid, such as water. In particular, particularly homogeneous emulsions with stable integration of the medium-chain triglycerides (MCTs) can be obtained on this basis.

In this context, it may in particular be provided that the composition comprises the emulsifier in an amount of at least 0.0001 wt. %, in particular at least 0.001 wt. %, preferably at least 0.01 wt. %, based on the composition. In addition, it may be provided according to the invention that the composition comprises the emulsifier in an amount of at most 5 wt. %, in particular at most 2.5 wt. %, preferably at most 1 wt. %, based on the composition. Further according to the invention, it may also be provided that the composition comprises the emulsifier in an amount in the range from 0.0001 wt. % to 5 wt. %, in particular in the range from 0.001 wt. % to 2.5 wt. %, preferably in the range from 0.01 wt. % to 1 wt. %, based on the composition.

The above quantities with respect to the optional emulsifier naturally relate only to the composition according to the invention as such. It goes without saying, however, that products based thereon, i.e. further products comprising the composition according to the invention (such as food, cosmetic and pharmaceutical products, etc.) may furthermore comprise additional emulsifiers or emulsifier components (such as surfactants, etc.), so that in these further products comprising the composition according to the invention significantly higher total amounts of emulsifier (e.g. from 7 wt. % to 20 wt. % in these further products) may be used to 20 wt. % in these formulation classes) may be present (such as in shampoos, shower gels, etc.).

According to the invention, the emulsifier can be an amphoteric, anionic, cationic or non-ionic emulsifier.

In the context of the present invention, it may in particular be provided that the emulsifier is selected from the group of fatty acid glycerides, in particular mono- and diglycerides of fatty acids, and their salts; citric acid esters of fatty acid glycerides, in particular citric acid esters of mono- and diglycerides of fatty acids, and their salts; tartaric acid esters and mono- and diacetyltartaric acid esters of fatty acid glycerides, in particular tartaric acid esters and diacetyltartaric acid esters of mono- and diglycerides of fatty acids, and their salts; acetic acid esters of fatty acid glycerides, in particular acetic acid esters of mono- and diglycerides of fatty acids, and their salts; lactic acid esters of fatty acid glycerides, in particular lactic acid esters of mono- and diglycerides of fatty acids, and their salts; mixed acetic and tartaric acid esters of fatty acid glycerides, in particular mixed acetic and tartaric acid esters of mono- and diglycerides of fatty acids, and their salts; polyglycerol fatty acid esters, in particular polyglycerol mono-, di- and poly-fatty acid esters, preferably polyglycerol polyricinoleate; sorbitan esters, in particular polyoxyethylene sorbitan esters, preferably polyoxyethylene sorbitan monoesters, preferably of fatty acids, in particular polyoxyethylene sorbitan monolaurate, polyoxyethylene sorbitan monooleate, polyoxyethylene sorbitan monopalmitate, polyoxyethylene sorbitan monostearate and polyoxyethylene sorbitan tristearate; ethoxylated fatty alcohols; ethoxylated fatty acid glycerides, in particular ethoxylated mono- and diglycerides of fatty acids, and their salts; alkyl polyglucosides and sugar glycerides, in particular cetearyl glucosides; sugar esters of fatty acids, in particular sucrose esters of fatty acids; modified starches; modified celluloses; quaternary ammonium compounds (ester quats); lecithins; phospholipids; alkyl glucose sesquistearates, in particular methyl glucose sesquistearate; fatty alcohols; fatty acids and their salts and esters; siloxanes and silicones; dimethicones and modified dimethicones, in particular PEG/PPG dimethicones; alkali metal alkyl sarcosinates, in particular sodium alkyl sarcosinates, preferably alkali metal lauryl sarcosinates; amino acid acyl esters and their salts; POE/PPG copolymers; phosphate esters (phosphoric acid esters) and their salts, in particular cetyl phosphates, preferably alkali metal cetyl phosphates, preferably potassium cetyl phosphate; polyacrylates; castor oils and modified castor oils, in particular PEG castor oils and PEG castor oil fatty acid esters and difatty acid esters; ethoxylated alkyl sulphates and alkyl ether sulphates; and alkyl ether sulphates; and combinations and mixtures thereof.

In particular, it may be provided according to the invention that the emulsifier is bound and/or fixed to the particles of the composition; and/or that the composition comprises the emulsifier in a form bound and/or fixed to the particles.

According to the invention, however, it may also be provided that the emulsifier is in particulate form and/or is in the form of emulsifier particles. In particular, the composition may comprise the emulsifier in the form of separate, in particular unbound and/or free, emulsifier particles.

The composition according to the invention may also be characterized by the following properties:

For example, the composition can be designed to be free-flowing or flowable in a preferred manner. This is accompanied by particularly good processing properties. In particular, good incorporation or incorporation of the composition into liquids, such as water, can be achieved on this basis. In particular, the dosing properties are also improved accordingly.

This also improves the preferably homogeneous incorporation into compositions in powder form.

Furthermore, it may be provided according to the invention that the composition according to the invention is at least substantially free of lumps or macroagglomerates. This also improves the behavior during the formation of dispersions or emulsions. In particular, no lump formation or the like occurs on this basis in the resulting dispersions or emulsions. This also improves the preferably homogeneous incorporation into compositions in powder form.

Furthermore, according to the invention, it may in particular be provided that the composition according to the present invention is at least substantially free of particle clumping or aggregation, in particular particle macroagglomerates, with sizes above 5 mm, in particular above 4.5 mm, preferably above 4 mm, particularly preferably above 3.5 mm. In particular, against this background, the particles of the composition behave according to the invention in such a way that the particles of the composition are at least substantially not agglomerated to form such particle clumps or clusters, in particular particle macroagglomerates. This increases the flowability and pourability and improves the formation of dispersions or emulsions. Furthermore, this also improves the preferably homogeneous incorporation into compositions in powder form.

Furthermore, the composition according to the invention is also characterized by a defined residual moisture: A defined residual moisture is also associated in particular with a further improved pourability or flowability of the composition. In addition, a defined or low residual moisture also counteracts lump formation during storage of the composition according to the invention, so that the composition according to the invention also has a high (storage) stability on this basis. In addition, the composition according to the invention also has an improved emulsifying capacity as a result of the defined residual moisture.

Thus, it may in particular be provided that the composition according to the invention has a residual moisture content of at most 5 wt. %, in particular at most 4 wt. %, preferably at most 3 wt. %, based on the composition.

In addition, the composition may have a residual moisture content of at least 0.1 wt. %, in particular at least 0.25 wt. %, preferably at least 0.5 wt. %, based on the composition.

Furthermore, the composition may have a residual moisture content in the range from 0.1 wt. % to 5 wt. %, in particular in the range from 0.25 wt. % to 4 wt. %, preferably in the range from 0.5 wt. % to 3 wt. %, based on the composition.

The composition according to the invention is also characterized by defined particle properties:

Thus, according to the invention, it may be provided that the particles of the composition have a D90 particle size, in particular a D90 particle diameter, in the range from 0.01 mm to 5 mm, in particular in the range from 0.05 mm to 2 mm, preferably in the range from 0.1 mm to 1 mm, particularly preferably in the range from 0.2 mm to 0.8 mm; in particular determined by light diffraction, preferably determined by laser diffraction according to ISO 13320:2020. The method of determination in this respect is well known to the skilled person as such, so that no further explanation is required in this respect.

Furthermore, the particles of the composition may have a D50 particle size, in particular a D50 particle diameter, in the range from 0.01 mm to 3 mm, in particular in the range from 0.05 mm to 2 mm, preferably in the range from 0.1 mm to 1 mm, particularly preferably in the range from 0.15 mm to 0.75 mm; in particular determined by light diffraction, preferably determined by laser diffraction according to ISO 13320:2020. The method of determination in this respect is well known to the skilled person as such, so that no further explanation is required in this respect.

According to one embodiment according to the invention, it is in particular provided that the composition according to the invention is at least substantially free of a further and/or additional carrier material, in particular is at least substantially free of a further and/or additional carrier material for the medium-chain triglycerides (MCTs).

In particular, it may be provided according to the invention that the composition comprises at least substantially no further and/or additional carrier material, in particular at least substantially no further and/or additional carrier material for the medium-chain triglycerides (MCTs).

Preferably, the composition may be at least substantially free of a further and/or additional polysaccharide-based carrier material. According to the invention, it may be provided that the composition comprises at least substantially no further and/or additional polysaccharide-based carrier material.

In particular, it may be provided according to the invention that the composition according to the invention is at least substantially free of a further and/or additional polysaccharide-based carrier material based on maltodextrin and/or starch, in particular modified starch.

In particular, it may be provided that the composition is at least substantially free of a further and/or additional polysaccharide-based carrier material based on maltodextrin and/or starch, in particular modified starch.

In this respect, it may in particular be provided that the composition according to the invention is at least substantially free of a further and/or additional polysaccharide-based carrier material comprising or consisting of maltodextrin and/or modified starch, or at least substantially does not comprise such a carrier material.

Furthermore, the composition may be at least substantially free of a carrier material other than the gum arabic, in particular at least substantially free of a polysaccharide-based carrier material other than the gum arabic, preferably at least substantially free of a gum arabic other than the gum arabic.

In particular, it may be provided according to the invention that the composition comprises at least substantially no carrier material other than the gum arabic, in particular at least substantially no polysaccharide-based carrier material other than the gum arabic, preferably at least substantially no gum arabic other than the gum arabic.

According to the invention, it may in particular be provided that the composition according to the invention does not comprise any further polysaccharide-based carrier material other than the gum arabic.

According to the invention, it may in particular also be provided that the composition according to the invention comprises no further and/or no other protein, in particular no phosphoprotein, preferably no casein, apart from glycoprotein(s).

Furthermore, according to the invention, it may in particular also be provided that the composition according to the invention does not comprise any phosphoprotein(s), preferably no casein.

On this basis, particularly uniform properties of the composition according to the invention can be provided with regard to the finishing or fixation with the medium-chain triglycerides (MCTs), including high loading quantities, as well as with regard to the dispersibility or emulsifiability of the composition according to the invention. In particular, the disadvantages associated with the other carrier materials are overcome, and this with a simultaneously high loading capacity of the composition according to the invention with respect to the medium-chain triglycerides (MCTs). In addition, there is a high compatibility and good compatibility.

According to the present aspect, the present invention also relates to the composition according to the invention, in particular particulate composition, preferably composition in powder form, preferably composition in powder form comprising medium-chain triglycerides (MCTs), in particular for use in cosmetic, pharmaceutical or food products, preferably a composition as defined above, wherein the composition is obtainable or obtained according to the method according to the invention as defined below.

The present invention, both according to the first aspect of the present invention and according to the further aspects as described below, is thus associated with a plurality of advantages and special features which make the composition according to the invention unique and special, in particular highly efficient.

For further details on this aspect of the invention, reference can also be made to the following explanations of the further aspects of the invention, whereby these explanations apply equally to the present aspect of the invention.

A further object of the present invention—according to a second aspect of the present invention—is furthermore the food, cosmetic or pharmaceutical product comprising the composition according to the invention, as described above.

In this context, using the composition according to the invention, corresponding products or products can be provided which can have an equally high content of medium-chain triglycerides and which are in the form of stable dispersions or emulsions. The products according to the invention have excellent handling and application properties. In addition, the relevant foodstuffs using the composition according to the invention have excellent organoleptic properties and at the same time very good nutritional properties.

According to a third aspect of the present invention, a further object of the present invention is the use of the composition according to the invention, as defined above, in a food, cosmetic or pharmaceutical product or for the preparation of a food, cosmetic or pharmaceutical product.

With regard to the aforementioned object according to the second and third aspects of the present invention, it may in particular behave in this way in this respect.

Thus, it may be provided according to the invention,
that the food, cosmetic or pharmaceutical product comprises the composition in an amount of at least 0.5 wt. %, in particular at least 1 wt. %, preferably at least 3 wt. %, preferably at least 5 wt. %, relative to the product; or
that the food, cosmetic or pharmaceutical product comprises the composition in an amount of at most 90 wt. %, in particular at most 80 wt. %, preferably at most 70 wt. %, preferably at most 60 wt. %, based on the product; or
that the food, cosmetic or pharmaceutical product comprises the composition in an amount in the range from 0.5 wt. % to 90 wt. %, in particular in the range from 1 wt. % to 80 wt. %, preferably in the range from 3 wt. % to 70 wt. %, preferably in the range from 5 wt. % to 60 wt. %, based on the product.

In general, it may also be provided according to the invention that the food, cosmetic or pharmaceutical product comprises the composition together with at least one carrier (excipient), in particular physiologically compatible carrier (excipient). In this way, the products can be further customized or specially designed, while at the same time ensuring a high level of compatibility.

In addition, the article according to the second and third aspects of the present invention may also be characterized by the following properties or formations: Thus, the food, cosmetic or pharmaceutical product, in particular food product, may be a functional food, a novel food, a food additive, a food supplement, a dietetic food and/or dietary food, a power snack, an appetite suppressant or a strength and/or endurance sports supplement.

In addition, the food, cosmetic or pharmaceutical product, in particular cosmetic product, may be a personal care and/or body cleansing product, in particular a skin and/or hair care and/or hair cleansing product, a skin and/or hair care and/or hair cleansing product, a skin and/or hair cleansing product, a skin and/or hair cleansing product, a skin and/or hair cleansing product or a hair cleansing product. Skin and/or hair cleansing product, preferably a shower gel, hair shampoo, bath additive, soap, skin cream, lotion, gel, cream, sunscreen, repellent, face mask, shaving foam or soap, self-tanning product, hair removal product, baby care product, beard care product, wet wipe, hygiene product for the intimate area or the like; a dental and/or oral care product or a dental and/or oral cleansing product, preferably a cosmetic product. A dental and/or oral care product or oral cleansing product, preferably toothpaste, mouthwash, adhesive composition for tooth replacement products or the like; a hair treatment product, preferably hair coloring product, hair setting product, hair conditioner or the like; an application product for optical and/or decorative purposes, preferably color cosmetic, lip care product, such as lipstick or lip gloss, eye care product, such as mascara or eye shadow, facial make-up or rouge, nail polish or the like; a hair treatment product, preferably hair coloring product, hair setting product, hair conditioner or the like; an application product for optical and/or decorative purposes, preferably color cosmetic, lip care product, such as lipstick or lip gloss, eye care product, such as mascara or eyeshadow, facial make-up or A body odor-influencing product or fragrance product, preferably deodorant, perfume, eau de toilette or the like.

According to the invention, it may also be provided that the food, cosmetic or pharmaceutical product is a liquid, ointment, cream, paste, lotion, gel, spray or solid, powder, tablet, pill, capsule, coated tablet or the like and/or is formulated and/or designed as a liquid, ointment, cream, paste, lotion, gel, spray, solid, powder, tablet, pill, capsule, coated tablet or the like.

According to an embodiment according to the invention, it may also be provided that the food, cosmetic or pharmaceutical product is in the form of a dry formulation, in particular a powdery dry formulation. According to the invention, it can behave in particular in such a way that the dry formulation is introduced and/or stored and/or incorporated in a liquid, in particular water, preferably in the context of application and/or use, in particular on the user and/or consumer side. In this way, ready-to-use (end) products can thus be provided, in particular by the user or consumers themselves, whereby stable dispersions, in particular emulsions, are also obtained on this basis.

For further details of the second or third aspect of the invention described herein, reference can also be made to the explanations of the other aspects of the invention, these explanations applying equally to the present aspect of the invention.

Similarly, it is an object of the present invention—according to a fourth aspect of the present invention—the process according to the invention for preparing a composition, in particular a particulate composition, preferably a composition in powder form, preferably a composition in powder form comprising medium-chain triglycerides (MCTs), in particular a process for preparing a composition as defined above,
the process comprising the following method steps, in particular in the order of the method steps given below:
   (i) Provision and/or production of a preferably aqueous homogeneous dispersion, in particular a preferably aqueous homogeneous emulsion, which comprises gum arabic, in particular as polysaccharide-based carrier material on the one hand, and at least one medium-chain triglyceride (MCT), in particular at least one triglyceride of at least one linear saturated C₆, C₈, C₁₀ or C₁₂ carboxylic acid, on the other hand, in a preferably aqueous liquid carrier medium,
      wherein a gum arabic with a glycoprotein content, based on the gum arabic, of at least 4 wt. % and preferably in the range from 4 wt. % to 20 wt. % is used and wherein the triglyceride is used in an amount of at least 50 wt. % and preferably in the range from 50 wt. % to 90 wt. %, based on the dry weight of the dispersion, in particular emulsion; hereinafter
   (ii) drying, in particular spray drying, of the dispersion, in particular emulsion, obtained in method step (i), with at least substantially complete removal of the preferably aqueous liquid carrier medium, in particular so that a particulate composition, preferably a composition in powder form, preferably a composition in powder form comprising medium-chain triglycerides (MCTs), in particular a composition as described above, results.

As far as the method according to the invention is concerned, method step (i) can be carried out with the introduction of shear energy and/or shear forces. In particular, in method step (i), the homogenization, in particular the homogenization of the ingredients, can be carried out with the introduction of shear energy and/or shear forces, in particular wherein the introduction of shear energy and/or shear forces is carried out by means of high-pressure homogenization and/or by means of a preferably high-pressure homogenizer. In particular, a high-pressure homogenization, in particular by means of a high-pressure homogenizer, can be carried out in method step (i).

In this context, it may be provided according to the invention that the high-pressure homogenization is carried out with a pressure gradient of up to 2,500 bar, in particular up to 2,250 bar, preferably up to 2,000 bar. In particular, the high-pressure homogenization can be carried out with a pressure gradient in the range from 50 bar to 2,500 bar, in particular in the range from 75 bar to 2,250 bar, preferably in the range from 100 bar to 2,000 bar.

For example, high-pressure homogenizers based on piston/gap or piston/nozzle homogenizers can be used.

In particular, continuous process control with high throughput is possible within the scope of the present invention. In particular, continuous production of the composition according to the invention is possible.

According to an embodiment according to the invention, it may also be provided that in method step (i) a two-stage and/or multi-stage method is carried out, in particular wherein in method step (i) at first a mixing, in particular stirring, preferably with the introduction of mixing and/or stirring energy and/or stirring forces, of the ingredients is effected, in particular for the purpose of obtaining a (pre-)dispersion, in particular (pre-)emulsion, and subsequently a homogenization, in particular a homogenization of the ingredients, is carried out with the introduction of shear energy and/or shear forces, preferably high-pressure homogenization of the (pre-)dispersion, in particular (pre-)emulsion, in particular as defined above, in particular to obtain the preferably aqueous homogeneous dispersion, in particular the preferably aqueous homogeneous emulsion of the ingredients.

On the basis of the process control according to the invention, in method step (I) a finely divided homogeneous dispersion, in particular a finely divided homogeneous emulsion, is produced and/or provided. This is also advantageous with regard to subsequent drying, in particular spray drying (cf. method step (ii)). In particular, a composition according to the invention with defined particle properties or sizes can also be provided on this basis.

In general, in method step (i), the homogeneous dispersion, in particular the homogeneous emulsion, can be adjusted to a viscosity, in particular apparent viscosity, of at most 2,500 mPa-s, in particular at most 1,000 mPa-s, preferably at most 400 mPa-s, or have a viscosity in this respect, in particular determined according to DIN EN ISO 2555 and in particular at room temperature (20 °C) and a shear rate of 10 s⁻¹. The viscosity can be determined in particular using a rotational viscometer. The defined viscosity leads to particularly good behavior during drying or spray drying to obtain the composition according to the invention.

As far as the composition obtained in method step (ii) is concerned, it may comprise or consist of a plurality of individual particles. According to the invention, it is provided that the particles each comprise gum arabic as a polysaccharide-based carrier material.

In this context, it may in particular be provided that the gum arabic and/or the polysaccharide-based carrier material is provided with the medium-chain triglyceride (MCT), in particular with the triglyceride of at least one linear saturated C₆, C₈, C₁₀ or C₁₂ carboxylic acid, and/or in particular that the particles comprise the gum arabic and/or the polysaccharide-based carrier material and the medium-chain triglyceride (MCT) bound and/or fixed to the polysaccharide-based carrier material, in particular the triglyceride bound and/or fixed to the polysaccharide-based carrier material of at least one linear saturated C₆, C₈, C₁₀ or C₁₂ carboxylic acid.

Based on the process according to the invention, the particle size can be adjusted in method step (i) and/or in method step (ii).

In the process according to the invention, the particle size can be adjusted in particular as follows,
that the particles of the composition comprise a D90 particle size, in particular a D90 particle diameter, in the range from 0.01 mm to 5 mm, in particular in the range from 0.05 mm to 2 mm, preferably in the range from 0.1 mm to 1 mm, particularly preferably in the range from 0.2 mm to 0.8 mm; in particular determined by light diffraction, preferably determined using laser diffraction according to ISO 13320:2020; and/or
that the particles of the composition comprise a D50 particle size, in particular a D50 particle diameter, in the range from 0.01 mm to 3 mm, in particular in the range from 0.05 mm to 2 mm, preferably in the range from 0.1 mm to 1 mm, particularly preferably in the range from 0.15 mm to 0.75 mm; in particular determined using light diffraction, preferably determined by laser diffraction according to ISO 13320:2020;
   in particular wherein the particle size is adjusted in method step (i), in particular on the basis of and/or by means of homogenization, in particular high-pressure homogenization, and/or in method step (ii) on the basis of and/or by means of drying, in particular spray drying, preferably in method step (ii) on the basis of and/or by means of drying, in particular spray drying.

In addition, the residual moisture content of the composition can be adjusted in method step (ii) as part of the process according to the invention.

In particular, in method step (ii) the composition can be adjusted to a residual moisture content of at most 5 wt. %, in particular at most 4 wt. %, preferably at most 3 wt. %, based on the composition.

Furthermore, in method step (ii), the composition can be adjusted to a residual moisture content of at least 0.1 wt. %, in particular at least 0.25 wt. %, preferably at least 0.5 wt. %, based on the composition.

In addition, in method step (ii), the composition can be adjusted to a residual moisture content in the range from 0.1 wt. % to 5 wt. %, in particular in the range from 0.25 wt. % to 4 wt. %, preferably in the range from 0.5 wt. % to 3 wt. %, based on the composition.

With regard to the process according to the invention, further drying can be carried out after the drying carried out in method step (ii), in particular spray drying. In particular, further drying may be carried out downstream of the drying carried out in method step (ii), in particular spray drying. In this context, the further and/or subsequent drying can be carried out for the purpose of (further) adjusting the residual moisture content of the composition, in particular as defined above.

According to the invention, in method step (ii) the drying, in particular spray drying, can be carried out by means of atomization and/or by means of spraying of the dispersion, in particular emulsion, obtained in method step (i).

In addition, in method step (ii), drying, in particular spray drying, can be carried out by passing and/or spraying the dispersion, in particular emulsion, obtained in method step (i) through at least one nozzle, in particular under (high) pressure.

In the process according to the invention, at least one emulsifier can be added or used in method step (i) and/or in method step (ii), in particular in method step (i), in particular as defined above.

According to the invention, it is also possible to proceed in such a way that, after method step (ii) has been carried out, at least one emulsifier is added to the composition obtained, in particular as defined above, in particular by spraying on or mixing and/or incorporating.

For further details of the methods according to the invention according to this aspect of the invention, reference can also be made to the explanations of the further aspects according to the invention, these explanations applying equally to the present aspect of the invention.

In addition, it is also an object of the present invention—according to a fifth aspect of the present invention—to provide the method for controlling and/or adjusting the loading, in particular loading amount, of a polysaccharide-based carrier material comprising gum arabic or formed therefrom with at least one medium-chain triglyceride (MCT), in particular with at least one triglyceride of at least one linear saturated C₆, C₈, C₁₀ or C₁₂ carboxylic acid, in particular for preparing a particulate and/or powder composition comprising medium-chain triglycerides (MCTs), preferably for preparing a composition as described above, the loading, in particular loading amount, being controlled and/or adjusted as a function of the glycoprotein content of the polysaccharide-based carrier material.

In this context, the gum arabic may have a glycoprotein content, based on the gum arabic, of at least 4 wt. % and preferably in the range from 4 wt. % to 25 wt. %.

In addition, the loading, in particular the loading amount, of triglyceride(s) may be at least 50 wt. % and preferably in the range of 50 wt. % to 90 wt. %, based on the total of polysaccharide-based carrier material and triglyceride(s), in particular based on the composition.

For further details of this aspect of the invention, reference can also be made to the explanations of the further aspects according to the invention, these explanations applying equally to the present aspect of the invention.

In addition, a further object of the present invention—according to a sixth aspect of the present invention—is the use according to the invention of the glycoprotein content of a polysaccharide-based carrier material comprising or formed from gum arabic for controlling and/or adjusting the loading, in particular the amount of loading, of the polysaccharide-based carrier material with at least one medium-chain triglyceride (MCT), in particular with at least one triglyceride of at least one linear saturated C₆, C₉, C₁₀ or C₁₂ carboxylic acid, in particular for the purpose of preparing a particulate and/or powder composition comprising medium-chain triglycerides (MCTs), preferably for the purpose of preparing a composition according to the invention as described above.

In this context, the gum arabic may have a glycoprotein content, relative to the gum arabic, of at least 4 wt. % and preferably in the range from 4 wt. % to 25 wt. %.

In addition, the loading, in particular the loading amount, of triglyceride(s) may be at least 50 wt. % and preferably in the range of 50 wt. % to 90 wt. %, based on the total of polysaccharide-based carrier material and triglyceride(s), in particular based on the composition.

For further details of this aspect of the invention, reference can also be made to the explanations of the further aspects according to the invention, these explanations applying equally to the present aspect of the invention.

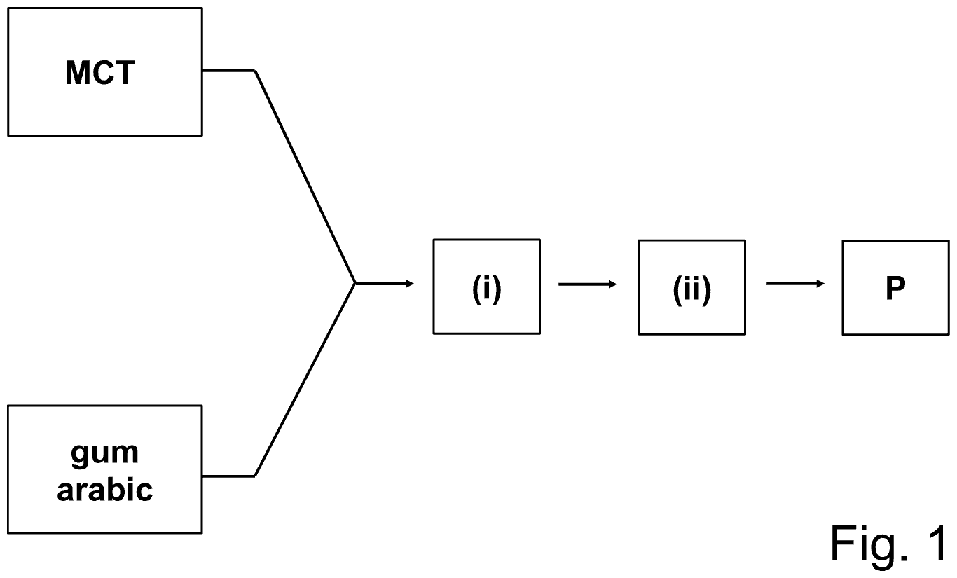

In the following, the present invention is explained in more detail with reference to a FIGURE illustration (FIG. 1) for further description, in particular of the method according to the invention for producing the composition according to the invention. Further advantages, properties, aspects and features of the present invention are also shown in connection with the following explanation of the process according to the invention, which is, however, in no way limiting with respect to the present invention.

In the only FIGURE representation, FIG. 1 shows a schematic representation or overview based on a flow chart of a process according to the invention for the preparation of a composition (composition P) according to the invention, in particular particulate composition P, preferably composition in powder form P, preferably composition in powder form P comprising medium-chain triglycerides (MCTs).

FIG. 1 shows the process according to one embodiment of the present invention, starting with the provision of the starting components in the form of the medium-chain triglyceride (MCT) and the special gum arabic used for the carrier material. FIG. 1 further illustrates the subsequent implementation of the process according to the invention with the sequence of method steps (i) and (ii) to obtain a composition P in this respect. In method step (i), in particular a preferably aqueous homogeneous dispersion, in particular a preferably aqueous homogeneous emulsion, is provided or produced, which comprises the gum arabic. which comprises the gum arabic, in particular as a polysaccharide-based carrier material, on the one hand and at least one medium-chain triglyceride (MCT), in particular at least one triglyceride of at least one linear saturated C₆, C₈, C₁₀ or C₁₂ carboxylic acid, on the other hand in a preferably aqueous liquid carrier medium,
a gum arabic having a glycoprotein content, based on the gum arabic, of at least 4 wt. % and preferably in the range from 4 wt. % to 20 wt. % being used in the process according to the invention, and the triglyceride being used in an amount of at least 50 wt. % and preferably in the range from 50 wt. % to 90 wt. %, based on the dry weight of the dispersion, in particular emulsion.

In method step (i), the process can be carried out in particular with the introduction of shear energy or shear forces. In particular, homogenization of the ingredients can be carried out in method step (i), for example by means of high-pressure homogenization.

FIG. 1 also illustrates the subsequent implementation of method step (ii) in method step (i), wherein in (ii) the drying, in particular spray drying, of the dispersion, in particular emulsion, obtained in method step (i) is carried out with at least substantially complete removal of the preferably aqueous liquid carrier medium, in particular so that the particulate composition P, preferably composition in powder form P, preferably composition in powder form P comprising medium-chain triglycerides (MCTs), in particular a composition P as defined above, results.

In method step (ii), the particle size of the particles on which the composition P according to the invention is based and the residual moisture content of the composition P can in particular also be adjusted.

Based on the method according to the invention, corresponding compositions P according to the invention are thus provided with improved properties in this respect, as stated above.

Further embodiments, modifications and variations as well as advantages of the present invention are readily apparent and realizable to the skilled person upon reading the description, without departing from the scope of the present invention.

The following examples are merely illustrative of the present invention, but without limiting the present invention thereto.

### Examples

In the context of the following working examples or studies, the outstanding properties of compositions according to the invention are illustrated, also with respect to providing high loading levels of medium chain triglycerides (MCTs). In this respect, the applicant can show that the use of a special gum arabic as a polysaccharide-based carrier material for the medium-chain triglycerides (MCTs), namely that the gum arabic has a high glycoprotein content in a special way, namely of at least 4 wt. %, based on the gum arabic, enables high loading levels or high contents of medium-chain triglyceride (MCT), according to which the composition or the particles, based on the composition or the particles, can have a triglyceride content of at least 50 wt. %. In detail:

### a) Preparation of MCT Compositions According to the Invention

The compositions according to the invention can be prepared starting from the ingredients or components forming the composition, in particular as follows.

In particular, it is possible to proceed in such a way that a preferably aqueous homogeneous dispersion, in particular a preferably aqueous homogeneous emulsion, is first prepared using the polysaccharide-based carrier material based on the special gum arabic and the medium-chain triglycerides using water. For example, a so-called “blending” can be carried out for this purpose. Water is first added, to which the gum arabic is added, whereby a (stirring) mixing of these components is carried out. The medium-chain triglyceride or MCT oil is then added and mixed to obtain a premix or “pre-blend”. This pre-blend is then subjected to or fed into a high-pressure homogenization process and homogenized on this basis. The resulting emulsion is then spray-dried. In order to obtain optimum results, the emulsion to be sprayed is adjusted to a specific viscosity, also depending on the spray nozzles used for spray drying. In the course of manufacture, in particular during spray drying, the particulate or composition in powder form obtained on this basis according to the invention is also adjusted to a defined residual moisture content, which should generally not exceed 5 wt. %, based on the composition.

### b) Preparation and Testing of MCT Compositions

On the basis of the manufacturing information given in section a), compositions according to the invention (compositions C, D, E) and corresponding comparative compositions (compositions A, B) are provided and investigated with regard to their properties, as stated below.

In the respective compositions, the content of glycoprotein and the corresponding loading of the composition or the particles with the medium-chain triglycerides (MCT) are adjusted or varied with respect to the gum arabic in accordance with the following table. On the basis of the corresponding combinations of values for the glycoprotein content on the one hand and the MCT loading on the other, as also stated in the following table, powders which are stable in storage and capable of emulsification can in principle be obtained, the compositions according to the invention having improved properties in this respect despite the higher MCT loading, as also stated below.

The present compositions are tested or investigated with regard to various parameters, namely with regard to (I) the emulsifiability in water at room temperature, (ii) the optical quality and quality of the emulsion obtained in each case and (iii) the stability of the emulsions obtained over a period of 10 to 15 min. The evaluation is summarized on the basis of the above parameters according to the school grading system (1=very good to 6=unsatisfactory). The results are shown in the table below.

TABLE
Properties of compositions comprising MCT

| | | MCT loading, | |
|---|---|---|---|
| | | based on the | |
| | Glycoprotein content, | composition or | |
| | based on gum arabic | particles | |
| Composition | [wt. %] | [wt. %] | Evaluation |
| A | 2 | 40 | 4 |
| B | 2.5 | 45 | 3-4 |
| C | 4 | 60 | 1-2 |
| D | 8 | 71 | 1 |
| E | 10 | 76 | 1 |

With the compositions C, D and E according to the invention, excellent properties are obtained overall with regard to the evaluation criteria examined, and this with simultaneously very high loading with the medium-chain triglycerides. On this basis, corresponding food, pharmaceutical or cosmetic products can therefore also be obtained using the compositions according to the invention with a high content of MCTs and a homogeneous emulsion structure with simultaneously high storage stability.

### c) Studies on the Residual Moisture Content of MCT-Comprising Compositions

On the basis of the table given in section b), corresponding compositions with varying residual moisture content are produced. For a respective residual moisture content which does not exceed 5 wt. %, based on the respective composition, the best overall properties with regard to storage time or (storage) stability and emulsion behavior are determined for the respective compositions. With a respective residual moisture content of more than 5 wt. %, the relevant properties are impaired. In particular, lump or agglomerate formation is also generally observed after a defined storage time, although this is significantly lower for the compositions C, D and E according to the invention than for the comparative compositions A and B. Thus, the degree of agglutination for the compositions according to the invention does not exceed the value of 2%, whereas this value for the comparative compositions is above 7% in each case. At respective residual moisture contents of less than 0.1 wt. %, there is a deterioration in the properties of the composition as a whole; in particular, the compositions tend to form a certain amount of dust, and the formation of emulsions when incorporated into water is also impaired. However, the compositions C to E according to the invention exhibit better properties in this respect than the comparative composition A or B, even for very low residual moisture contents.

### d) Preparation of a Skin Care Cream Using MCT-Comprising Compositions According to the Invention

On the basis of the compositions C to E according to the invention, respective skin protection or skin care creams are produced, namely on the basis of three phases which are combined as described below. The first phase comprises the respective composition according to the invention and, in particular, oil, fat or wax components. The second phase is based on water, glycerol monoester and zinc sulphate. The third phase comprises a tocopherol and perfume or fragrance components. To produce the respective cosmetic products, the first phase and the second phase are first heated. The second phase is then added to the first phase while stirring, followed by homogenization. The resulting phase is then cooled, followed by the addition of the third phase and further homogenization. This results in corresponding skin protection or skin care creams as cosmetic products using the respective compositions according to the invention. The cosmetic products are storage-stable and have a high content of medium-chain triglycerides. This results in excellent application properties, also with regard to application to the skin. They also have excellent skin care properties.

The above explanations show that the compositions according to the invention and products or articles based thereon are associated with advantageous properties, in particular with regard to the formation of stable emulsions, high storage stability, high MCT contents and good application and handling properties.

1-68. (canceled)

69. A composition in powder form wherein the composition comprises medium-chain triglycerides,
wherein the composition comprises or consists of a plurality of individual particles,
wherein the particles each comprise at least one polysaccharide-based carrier material, wherein the polysaccharide-based carrier material is provided with at least one medium-chain triglyceride wherein the at least one medium-chain triglyceride is fixed to the polysaccharide-based carrier material;
wherein the polysaccharide-based carrier material is formed from gum arabic, wherein the gum arabic comprises a glycoprotein content, based on the gum arabic, in the range of from 4 wt. % to 25 wt. %, wherein the glycoprotein content is based the total content of all glycoproteins present in the gum arabic, and
wherein the composition comprises, based on the composition, a triglyceride content in the range of from 50 wt. % to 90 wt. %.

70. The composition according to claim 69,
wherein the composition is further characterized by at least one of the following features:
the gum arabic is of natural plant-based origin;
the gum arabic is of natural plant-based origin, wherein the gum arabic is based on a plant-based exudate of acacia trees;
the gum arabic is of natural plant-based origin, wherein the gum arabic is based on a plant-based exudate of acacia trees of Senegalia senegal.

71. The composition according to claim 69,
wherein the glycoprotein content of the gum arabic is controlled and set by selecting the plant-based material used for obtaining the gum arabic on the basis of an exudate with additional glycoprotein enrichment via fractionation.

72. The composition according to claim 69,
wherein the glycoprotein content is provided by macromolecules comprising at least one of a protein and a protein residue each comprising one or more carbohydrate groups attached thereto, wherein the carbohydrate groups are selected from the group consisting of monosaccharides, disaccharides, oligosaccharides and polysaccharides and combinations thereof.

73. The composition according to claim 69,
wherein the composition is further characterized by at least one of the following features:
the glycoprotein content is provided by glycoproteins having a weight average molecular weight M̅_{w} in the range of from 10⁴ g/mol to 10⁷ g/mol;
the glycoprotein content is provided by glycoproteins having a weight average molecular weight M̅_{w} in the range of from 10⁴ g/mol to 10⁷ g/mol, wherein 50% to 99% is provided by arabinogalactan proteins, based on the glycoprotein content;
50% to 99% of the glycoprotein content is provided by arabinogalactan proteins, based on the glycoprotein content.

74. The composition according to claim 69,
wherein the composition is further characterized by at least one of the following features:
the gum arabic comprises, as main constituent, at least one polysaccharide comprising at least one of L-arabinose, D-galactose, L-rhamnose and D-glucuronic acid;
the gum arabic comprises a polysaccharide content, based on the gum arabic, in the range of from 75 wt. % to 96 wt. %.

75. The composition according to claim 69,
wherein the medium-chain triglyceride is selected from the group consisting of triglycerides of at least one of C₆ carboxylic acid, C₈ carboxylic acid, C₁₀ carboxylic acid and C₁₂ carboxylic acid and combinations thereof.

76. The composition according to claim 69,
wherein the medium-chain triglyceride is selected from the group consisting of triglycerides of at least one of linear saturated C₆ carboxylic acid, linear saturated C₈ carboxylic acid, linear saturated C₁₀ carboxylic acid and linear saturated C₁₂ carboxylic acid and combinations thereof.

77. The composition according to claim 69,
wherein the medium-chain triglyceride is selected from the group consisting of triglycerides of C₈ carboxylic acids, triglycerides of C₁₀ carboxylic acids and mixed triglycerides of C₈ and C₁₀ carboxylic acids and combinations thereof.

78. The composition according to claim 69,
wherein the composition is further characterized by at least one of the following features:
the medium-chain triglyceride is a triglyceride of a C₈ carboxylic acid;
the medium-chain triglyceride is tricaprylin;
the medium chain triglyceride is a mixed triglyceride of C₈ and C₁₀ carboxylic acids;
the medium chain triglyceride is a mixed triglyceride of linear saturated C₈ and C₁₀ carboxylic acids;
the medium chain triglyceride is a mixed triglyceride of C₈ and C₁₀ carboxylic acids, wherein the medium-chain triglyceride has a C₈:C₁₀ ratio in the range of (30%-90%):(70%-10%);
the medium chain triglyceride is a mixed triglyceride of linear saturated C₈ and C₁₀ carboxylic acids, wherein the medium-chain triglyceride has a C₈:C₁₀ ratio in the range of (30%-90%):(70%-10%).

79. The composition according to claim 69,
wherein the composition comprises a ketogenic index, calculated as the theoretical number of millimoles of 3-hydroxybutyric acid per one gram of the composition, in the range of from 0.5 mmol/g to 50 mmol/g.

80. The composition according to claim 69,
wherein the composition further comprises at least one emulsifier.

81. The composition according to claim 69,
wherein the composition further comprises at least one emulsifier;
wherein the composition contains the emulsifier in an amount in the range of from 0.0001 wt. % to 5 wt. %, based on the composition, and
wherein the emulsifier is selected from the group consisting of ionic emulsifiers, non-ionic emulsifiers and amphoteric emulsifiers and combinations thereof.

82. The composition according to claim 69,
wherein the composition further comprises at least one emulsifier;
wherein the emulsifier is selected from the group consisting of emulsifiers fixed to the particles of the composition and emulsifiers in the form of separate emulsifier particles and combinations thereof.

83. The composition according to claim 69,
wherein the composition is further characterized by at least one of the following features:
the composition is free-flowing;
the composition is flowable;
the composition is at least substantially free from clumping;
the composition is at least substantially free of particle clumps or clusters with sizes above 5 mm;
the composition comprises a residual moisture content in the range of from 0.1 wt. % to 5 wt. %;
the particles of the composition comprise a D90 particle diameter in the range of from 0.01 mm to 5 mm, determined using light diffraction;
the particles of the composition comprise a D50 particle diameter in the range of from 0.01 mm to 3 mm, determined by light diffraction.

84. A product selected from the group consisting of foods, cosmetics and pharmaceutical, wherein the product comprises a composition according to claim 69.

85. A method for producing a medium chain triglycerides comprising composition according to claim 69,
wherein the method comprises the following method steps (i) and (ii) in the order of the method steps given below:
(i) a first step of providing a homogeneous dispersion which comprises gum arabic as a polysaccharide-based carrier material and at least one medium-chain triglyceride in a liquid medium,
wherein the gum arabic used has a glycoprotein content, based on the gum arabic, in the range of from 4 wt. % to 25 wt. % and wherein the triglyceride is used in an amount in the range of from 50 wt. % to 90 wt. %, based on the dry weight of the dispersion;
(ii) subsequently, a step of drying of the dispersion obtained in method step (i) while at least substantially completely removing the liquid medium, so as to yield a composition in powder form according to claim 69.

86. A method for controlling the amount of loading of a polysaccharide-based carrier material comprising gum arabic with at least one medium-chain triglyceride for producing a particulate composition in powder form according to claim 69, wherein the composition comprises medium-chain triglycerides,
wherein the amount of loading is controlled and set as a function of the glycoprotein content of the polysaccharide-based carrier material,
wherein the gum arabic comprises a glycoprotein content, based on the gum arabic, in the range of from 4 wt. % to 25 wt. %, wherein the glycoprotein content is based on the total content of all glycoproteins present in the gum arabic, and
wherein the loading amount, of triglycerides is in the range of from 50 wt. % to 90 wt. %, based on the composition.
